(19) United States
(12) Patent Application Publication
(10) Pub. No.: US 2009/0105798 A1
(43) Pub. Date: Apr. 23, 2009

(54) HANDLE FOR STEPWISE DEPLOYMENT

(75) Inventor: INA KOCH, Villingen-Schwenningen (DE)

Correspondence Address: FULWIDER PATTON, LLP (ABBOTT), 6060 CENTER DRIVE, 10TH FLOOR, LOS ANGELES, CA, 90045, US

(73) Assignee: ABBOTT LABORATORIES VASCULAR ENTERPRISES LIMITED, Dublin (IE)

(21) Appl. No.: 11/874,016

(22) Filed: Oct. 17, 2007

(51) Int. Cl. A61F 2/06

(52) U.S. Cl. 623/1.11

(57) ABSTRACT

A system for controlled deployment of a prosthesis, in which a sheath is retracted from a prosthesis in a body vessel or duct. The system includes a handle for deploying the prosthesis, the handle comprises a base member fixed to a catheter, and a slide member for sliding past the base member, the slide member being fixed to an end of a retraction member. The slide member includes a serrated gear rod, and the handle is configured such that, when activated, the gear rod moves in relation to the base member by no more nor less than the length of one serration on the gear rod.

### BACKGROUND OF THE INVENTION

The present invention relates to an implant delivery or deployment system. More specifically, the invention relates to a deployment system that uses a handle operable under automatically controlled conditions to retract the retractable outer sheath and deploy a medical implant for minimally invasive application, such as an endovascular stent graft, vena cava filter, self-expanding stent, balloon expandable stent, angioplasty balloon, and the like.

Delivery systems for deploying a medical implant, such as an endovascular stent graft, vena cava filter, self-expanding stent, balloon expandable stent or the like, are known in the field of medical technology. These medical devices have many uses and applications. In particular, a stent is a prosthesis or implant which is generally tubular with openings formed therein, and which is expanded radially in a vessel or lumen to improve and reinforce the vessel's patency. Stents are widely used in body lumens, body canals, ducts or other body vessels. A self-expanding stent is a stent which expands from a first compressed condition during delivery to a second expanded condition when released from the delivery device, where the stent exerts outward radial force on a portion of the body vessel to improve and maintain patency. One common self-expanding stent is manufactured of Nitinol, a nickel-titanium shape memory alloy, which can be formed and annealed, compressed from its original shape and held at a low temperature, and recalled to its original shape with heating, such as when deployed at body temperature in the body.

One important need felt in the art for delivering a stent or other prosthesis/implant is the ability to retract the outer sheath in a controlled and precise manner, to enable the physician to accurately determine proper positioning of the stent as well as track the retraction of the outer sheath. Although devices exist in the art for retracting the outer sheath, these are typically complex and expensive to manufacture, having no safeguard to prevent accidental instantaneous or early deployment of the entire stent. For example, such devices may allow the physician, in one complete movement of his hand, to deploy substantially the entire stent. Such devices are prone to accident, in that the physician may inadvertently apply a movement that is too large for the intended purpose. Accordingly, there is also a need for a system that is simple to fabricate and economical to manufacture, allowing for disposal after use, in which system the operator is compelled to take multiple separate steps to deploy the prosthesis, and which does not allow for a single step or movement to be taken that may deploy substantially the entire prosthesis, or an excessively large part thereof. The present invention addresses these and other needs.

### SUMMARY OF THE INVENTION

In a preferred embodiment there is described a system for the controlled delivery of a prosthesis or implant in a body vessel or duct. The delivery system includes an elongate core cylinder, or catheter, for delivering a prosthesis to a predetermined location in the duct. A prosthesis surrounds the core cylinder, and a sheath surrounds the prosthesis. A handle for deploying the prosthesis is provided, the handle comprising a base member connected to the core cylinder. The base member has an elongate axis. A slide member, operably attached to the sheath, is provided, the slide member being moveable in relation to the base member, whereby, when the slide member is moved in relation to the base member, the sheath is moved in relation to the prosthesis.

An elongate gear rod defining a plurality of serrations, is provided, each serration being of equal length, the gear rod being attached to the slide member. An activation element, or button, is pivotally connected to the base member, and a driving element extends from the activation element. Under this configuration, pivotal depression of the activation element causes the driving element to engage one of the serrations on the gear rod and to move the gear rod proximally in relation to the base member, thereby incrementally retracting the sheath from the prosthesis. In an important aspect of the invention, the activation element is dimensionally configured in relation to the base member and the gear rod so that proximal movement of the gear rod caused by a single depression of the activation element cannot exceed the length of a single serration. This structure has the advantage of permitting movement of the sheath in relation to the catheter of only a predetermined small amount for each depression of the activation element. This provides for greater control, and will not allow accidental hand movement by the operator to deploy an excessive amount of the prosthesis at one time.

In another aspect of the invention, the deployment system further includes a locking element, configured to prevent the gear rod from moving distally in relation to the base member. Preferably, the locking element extends substantially perpendicular to the gear rod, and may define an opening for permitting passage of the gear rod. A biasing element is provided, configured to urge the locking element in a direction perpendicular to the gear rod. Preferably, the locking element is configured to be manually moved, against the urging of the biasing element, so as to unlock the gear rod and thereby to permit the gear rod to move distally in relation to the base member. These aspects allow the locking element to be temporarily unlocked for repositioning of the sheath.

In yet another aspect of the invention, the slide member may be operably attached to the sheath via a retraction member that includes a tubular portion surrounding the core cylinder. This feature allows for a reduced effective diameter of the delivery system.

In yet a further aspect of the invention, the base member may include a circular cylinder, and the slide member may include a piston slideably fitted within the circular cylinder.

Finally, the activation element may be biased to a starting position by a leaf spring that defines an opening for passage of the gear rod.

In a different aspect, the invention includes a method of retracting a sheath to uncover a prosthesis mounted on a catheter. The method includes attaching the catheter to a base member having a first elongate axis, the base member being positioned proximal to the catheter. A sheath is operably attached to a slide member having a second elongate axis, the slide member being slideable parallel to the first axis. An activation element pivotally attached to the base member is repeatedly depressed. The slide member is caused to move proximally, by no more than the length of a single serration on a gear rod attached to the slide member, with each depression of the activation element. After each depression, the activation element is withdrawn to a starting position, so that the sheath is incrementally moved proximally to uncover the prosthesis.

In a further aspect, the gear rod is locked against distal movement in relation to the base member. In yet a further aspect, withdrawing the activation element after each depression includes withdrawing the activation element under the urging of a leaf spring. And further, causing the slide member to move proximally may include engaging the gear rod with a driving element attached to the activation element.

The present invention is applicable to deployment of devices that may be left behind in a vessel or duct, such as a stent, and also to devices that may be removed from the vessel or duct after deployment, such as a balloon on its own, not associated with a stent. Accordingly, the terms “implant” and “prosthesis” shall be used herein as not requiring that some structure be left behind in the vessel or duct after deployment, and will cover a balloon alone, removed after deployment.

These, and other features of the invention, will be disclosed more fully in the detailed description of the preferred embodiments that follow, and the drawings attached.

### BRIEF DESCRIPTION OF THE DRAWINGS

FIG. 1 is a schematic view, in partial section, depicting a controlled prosthesis delivery system having features of the present invention.

FIG. 2 is a sectional perspective view of a feature of a preferred embodiment of the above invention.

FIG. 3 is a sectional view taken substantially along the line 3-3 of FIG. 1.

FIG. 4 is a sectional view taken substantially along the line 4-4 of FIG. 1.

FIG. 5 is an enlarged detail identified within the circle marked “5” in FIG. 1.

### DETAILED DESCRIPTION OF THE PREFERRED EMBODIMENTS

With reference to the drawings, which are provided by way of exemplification and not limitation, there is described an apparatus for controlled stent delivery having features of the present invention.

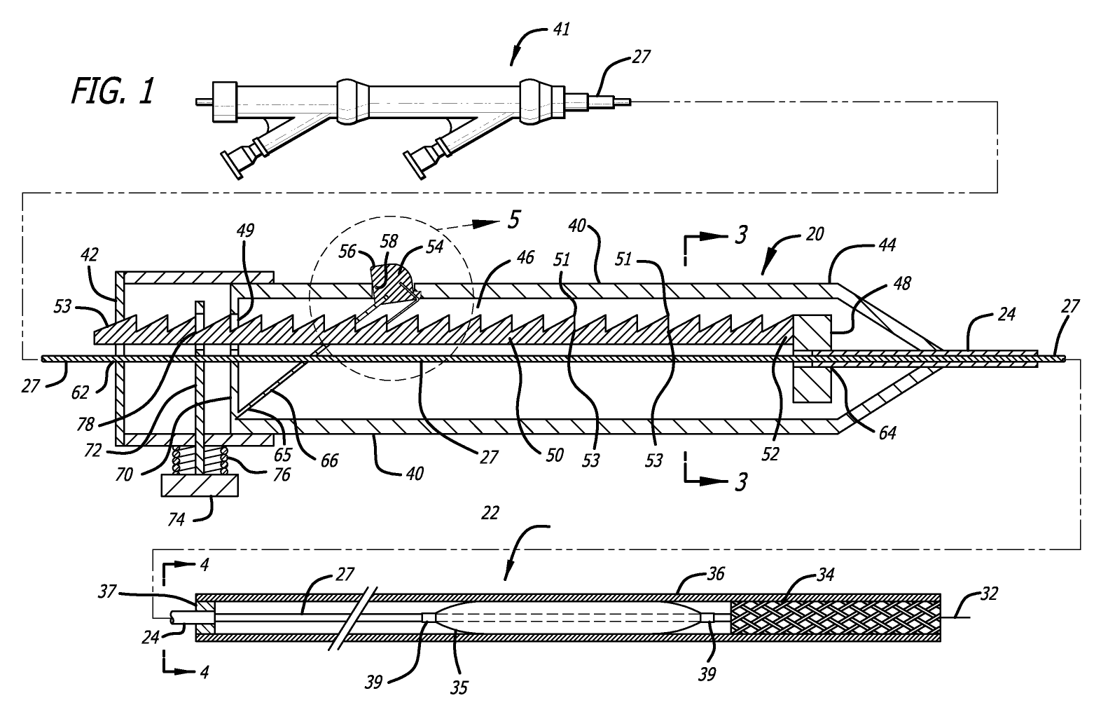

A preferred embodiment of the invention includes a release handle, generally identified by the numeral 20. (FIG. 1.) The release handle is adapted to operate in conjunction with a catheter 22. The catheter is configured to remotely deliver a prosthesis in a body vessel or duct.

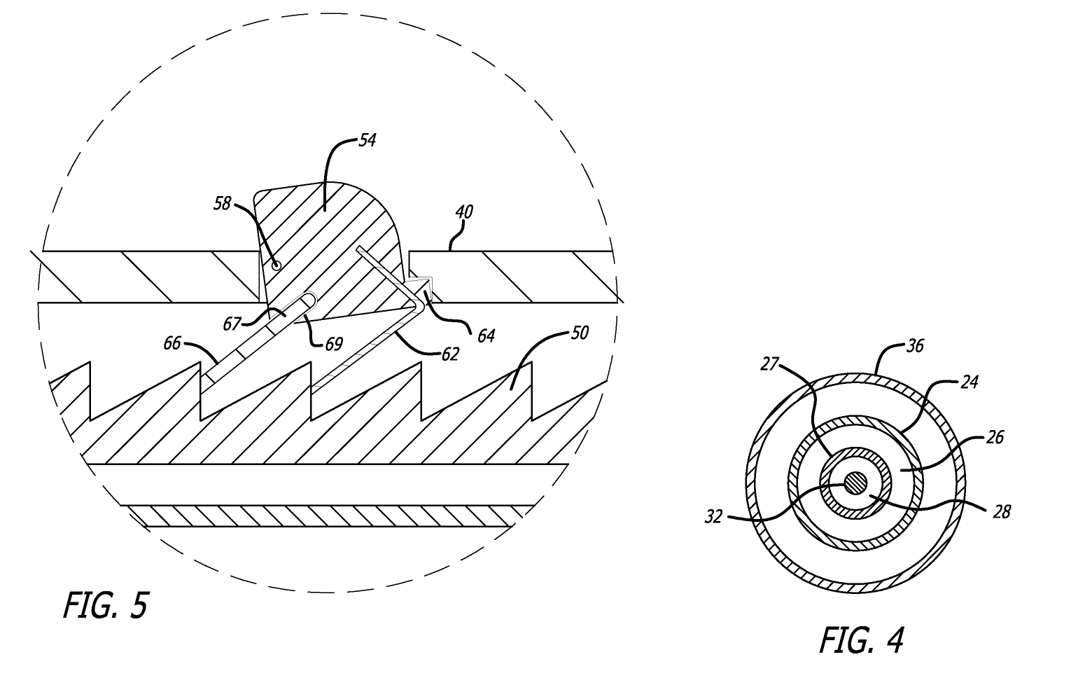

Catheters for remotely delivering a prosthesis are known, and may include a core cylinder 27 having an internal lumen 28. (FIG. 4.) A guidewire 32 may be inserted within the lumen 28, according to known usage wherein the guidewire is first threaded into a body vessel (not shown), whereafter the catheter 22 is run up into the body vessel over the guidewire 32. At the distal end of the catheter, a prosthesis may be positioned to surround the catheter. In FIG. 1 it is shown that the prosthesis may be a self-expanding stent 34, although the present invention may work in conjunction with other types of prostheses. Additionally, an inflatable balloon 35 may be positioned on the catheter for insertion within the stent after the stent is deployed, whereupon inflation of the balloon may position the stent at the correct diameter. An outer retractable sheath 36 is positioned to surround the prosthesis or stent 34 so that, when the catheter 22 is introduced into a body vessel, the stent is confined and protected within the sheath 36 where it will not cause trauma to the walls of the body vessel nor prematurely deploy. Subsequent retraction of the sheath 36 to uncover the stent allows the stent to expand. Once the stent is correctly positioned within the body vessel (which positioning may include balloon inflation), the catheter 22 is withdrawn, leaving the stent to reinforce the body vessel and maintain its patency.

In a preferred embodiment of the present invention, a retraction member 24 may be provided for exerting a retraction force to the sheath 36. The distal end of the retraction member 24 is connected to the sheath 36 at a connection point 37, so that any proximal displacement imparted to the retraction member 24 is also imparted to the sheath 36. The proximal end of the retraction member is connected to the release handle 20 as described in more detail below. The connection point 37 may be by way of an annulus, configured to transfer tensile load from the retraction member 24 having a smaller diameter, to the sheath 36, having a larger diameter.

The retraction member 24 may be a rod, wire or hypotube, that can be made of a metal or polymeric material. In the embodiment disclosed in the figures, the retracting member 24 is positioned to surround the core cylinder 27. (FIG. 4.) The retracting member lumen 26 may additionally carry flushing fluid for purging and cleaning the catheter. If the retracting member 24 is a hypotube, the hypotube can be configured to carry the purging fluid. Additionally, purging fluid may be introduced into the catheter 22 generally via a standard luer connector 41 positioned at the proximal end of the catheter.

The retractable sheath 36 may be flexible or rigid, but in a preferred embodiment is flexible to facilitate delivery of the catheter 22 to remote portions of the body vessel. If an inflatable balloon 35 is provided, the sheath 36 may also cover the balloon. Radiopaque markers 39 may be positioned along the length of the catheter to mark the positions of the balloon 35 and the stent 34 in order to facilitate delivery of the stent using known methods of radiography.

Turning now to FIGS. 1-5, there is exemplified a release handle 20 having features of the present invention for activating the catheter 22 to deploy the prosthesis 34. A base member is provided for grasping and protecting the components of the handle. In a preferred embodiment, the base member is a cylinder 40 having an elongate axis with a proximal end 42 and a distal end 44 and an internal bore 46. The cylinder 40 is provided for manually grasping the control handle 20 and for protecting the inner workings of the handle. A slide member is configured to slide along the base member. Where the base member is a cylinder, the slide member is a piston 48 (FIGS. 1 and 3) configured to slidingly reciprocate within the bore 46 of the cylinder 40. Preferably, the piston is configured to fit snugly within the bore of the cylinder to eliminate wobble, and may be lubricated.

In order to configure the handle 20 so that the sheath 36 may be withdrawn from its position covering the prosthesis in a slow and controlled manner, without the possibility of a sudden withdrawal of the entire sheath or a substantial portion of the sheath from the prosthesis by accidental movement of the user, the following structure is described:

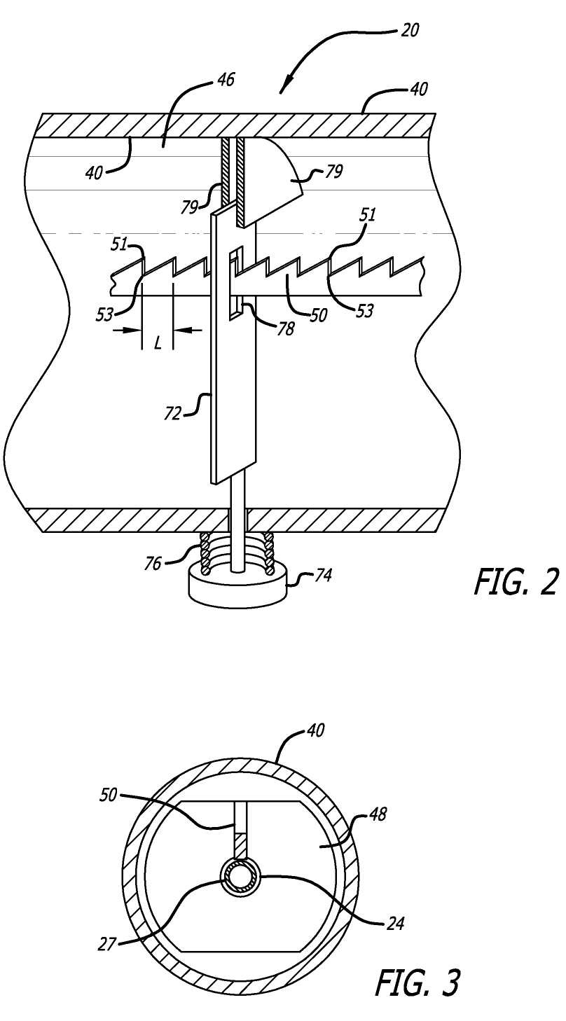

An elongate serrated gear rod 50 is connected at its distal end 52 to the piston 48. At its proximal end 53, the gear rod 50 passes through an opening 49 in the proximal end 42 of the cylinder. As a result, the gear rod spans between the piston 48 and the proximal end of the cylinder. The gear rod includes a plurality of peaks 51 and valleys 53, and the length between one peak and the next is a constant distance “L” as indicated in FIG. 2.

An activation element 54, or button, is provided, with its proximal end 56 pivotally attached by a pin 58 to the cylinder 40. As a result, the activation element may rotate about the pin. Attached to the activation element 54 is an elongate driving element 62 which is angled proximally so that, when the activation element 54 is manually depressed to rotate inwardly about the pin 58, the driving element 62 is configured to engage one of the serrations in the gear rod, and force the gear rod proximally within the bore 46 of the cylinder. The driving element 62 is preferably configured to be slightly flexible along its elongate axis, so that it may assume an appropriate deformed shape as it rotates with the activation element while engaged with the gear rod.

One aspect of the invention is that the activation element 54 is configured in relation to the base member 40 and the length “L” between serrations on the gear rod 50 such that, when the activation element is manually depressed by the user to the full extent of its travel, it will cause the gear rod to advance proximally in relation to the base member by the length “L” of only one serration before the activation element runs out of travel and must be retracted outwardly before it may be depressed again. Outward retraction of the activation element may be caused by a spring, which in a preferred embodiment is a leaf spring 66, cantilevered from a proximal end 65 thereof and attached to the activation element 54 at a distal end 67 thereof, as shown in FIGS. 1 and 5. The leaf spring 66 may define an opening 71 through which the gear rod 50 may pass. In a preferred embodiment, the distal end 67 of the leaf spring may be configured to slide freely within an opening 69 in the activation element 54, to permit unrestricted rotation of the activation element about the pin 58. Again in the preferred embodiment, the proximal end 65 of the leaf spring 66 forms a continuous connection with a vertical element 70, that may extend across the diameter of the cylinder 40, to provide a continuous moment bearing cantilever connection for the leaf spring. It will be appreciated that, under this arrangement, the leaf spring 66 will tend to restore the activation element 54 to an outwardly pivoted equilibrium position when the activation element is released after being depressed, and this action will in turn cause the driving member 62 to retract upwardly over a serration peak 51 in the gear rod 50, to drop into the next serration valley 53, ready for the next incremental or stepwise movement.

As noted, the activation element 54 and its driving member 66 are dimensionally configured in relation to the dimensions of the other elements of the handle 20 such that the gear rod 50 may not be advanced by more than the length “L” of a single serration each time the activation element is manually depressed to the maximum extent of its travel. With this limitation applied to the structure of the system, a user cannot accidentally cause the sheath to be withdrawn from the prosthesis too rapidly. Each small incremental retraction of the sheath must be made by the user as a separate intentional manual action, and this provides protection against accidental rapid deployment of the prosthesis in which the user may accidentally overextend a single manual movement.

In order to prevent the activation element 54 from retracting too far outwardly, which might disengage the activation element from the cylinder 40, a lip 64 (FIG. 5) may be provided on an edge of the activation element to engage with the cylinder and prevent outward movement beyond a desired degree of travel.

In order to control and prevent distal movement of the gear rod 50 relative to the cylinder 40, a vertically extending locking element 72 may be provided. The vertically extending locking element extends through the wall of the cylinder 40 and includes a knob 74 on the outside of the cylinder, attached at an extremity of the locking element. Between the knob 74 and the wall of the cylinder a biasing member, preferably a helical spring 76, may be positioned to urge the locking element 72 downwardly. The locking element defines an opening 78 through which the gear rod 50 snugly passes. Parallel retention plates 79 may be attached to the inner surface of the cylinder 40, and are configured to cause the locking element 72 to move up and down without being able to bend out of vertical alignment. When the handle is in use, proximal movement of the serrated gear rod 50 through the opening 78 causes the vertical locking element 72 to be urged upward against the bias of the spring 76 until the opening 78 passes over a peak 51 of a serration, whereupon it falls to the valley 53 of the next serration. Thus, the vertical locking element 72, by applying a ratchet effect, prevents the gear rod 50 from making large movements distally in relation to the cylinder 40, but permits incremental proximal movement under operation of the activation element 54. The locking element 72 also provides a confirmatory “click” sound and tactile vibration each time the peak 51 of a serration passes through the opening 78. Additionally, the vertical locking member 72 is manually operable from outside the handle. By manually depressing the knob 74 toward the center of the cylinder 40, the locking element 72 releases its restraining grip on the gear rod 50, and allows a user to realign the sheath by moving the gear rod distally in relation to the cylinder 40.

Turning now to the interaction between the catheter 22 and the release handle 20, the retraction member 24 is fixed to the piston 48 at a connection point 64 (FIG. 1), while the core cylinder 27 is fixed to the cylinder at a connection point 62. Thus, it will be appreciated, retraction (i.e. proximal movement) of the piston 48 in relation to the cylinder 40 will result in retraction of the retraction member 24 in relation to the core cylinder 27, and thus retraction of the sheath 36 in relation to the prosthesis 34.

The system for controlled delivery of a prosthesis may be assembled (or loaded) for use as follows. The piston 48 with attached gear rod may be positioned within the bore 46 of the cylinder 40 by threading the gear rod through the opening 78 of the locking member 72 until the gear rod is in a distal starting position (as exemplified in FIG. 1). Thereafter, the catheter 22 may be affixed to the handle, as described above, in which the core cylinder 27 is fixed to the cylinder 40 at connection point 62, and the retraction member 24 is fixed to the piston 48 at connection point 64. Connection points 62 and 64 may be achieved by adhesive, compressive, frictional, or similar means. It will be appreciated that it is not essential to connect the sheath to the piston via a retraction member, but the general objective is to operably connect the sheath to the piston whereby movement of the piston 48 in relation to the base member 40 causes movement of the sheath 36 in relation to the core cylinder 27 upon which the prosthesis is mounted. Connecting the sheath to the piston via a retraction member 24 as described is simply a preferred embodiment of the invention in that it permits a reduced diameter over much of the length of the catheter 22.

Turning now to actual operation of the system once it is loaded, the physician first inserts the catheter 22 with mounted prosthesis 34 into a body vessel (not shown) following a standard method known in the art. When the physician is satisfied that the prosthesis is in the correct position in relation to the body vessel and wishes to retract the sheath 36 to deploy the prosthesis 34, she depresses the activation element 54 a number of times, each time confirming the proper operation of the device by sensing the clicking action of the vertical locking element 72. At intervals as desired, the user may check the position of the prosthesis within the body vessel using the radiopaque markers 39 in a conventional way.

Following this technique, the gear rod 50 and piston 48 can be moved proximally along the length of the handle 20 under a controlled methodology, and the sheath 36 can be completely retracted from the prosthesis to permit deployment of the prosthesis. It will be appreciated that, under the dimensional configuration as described herein, each depression of the activation element will retract the sheath from the prosthesis by no more than the length of one serration on the gear rod 50, and thus the user cannot accidentally remove the sheath by an excessively large amount through accidental overextension of a single hand movement.

In a preferred embodiment, the cylinder 40 may be molded from plastic. The piston 48 may also be molded from plastic, but the gear rod 50, with its dimensionally precise serrations is preferably made of metal.

It will be appreciated that, in the manner described, the physician operator can control the retraction of the sheath 36 from the prosthesis 34 in short incremental stages. The structure described is extremely simple, it requires few parts, and is therefore inexpensive to manufacture so as to permit disposal of the entire unit after use. This provides an effective, yet inexpensive, solution to the problem of providing a system for deployment of a prosthesis in a body vessel, one over which the operator cannot accidentally lose control through a single excessive hand movement.

It will be realized that the foregoing preferred specific embodiments have been shown and described for the purpose of illustrating the functional and structural principles of this invention and are subject to change without departure from such principles. Therefore, this invention includes all modifications encompassed within the spirit and scope of the following claims.

We claim:

1. A deployment system for deploying a prosthesis in a duct, comprising:
an elongate core cylinder for delivering a prosthesis to a predetermined location in the duct;
a prosthesis surrounding the core cylinder;
a sheath surrounding the prosthesis; and
a handle for deploying the prosthesis, the handle comprising:
a base member connected to the core cylinder, the base member having an elongate axis;
a slide member operably connected to the sheath, the slide member being moveable in relation to the base member, whereby, when the slide member is moved in relation to the base member, the sheath is moved in relation to the prosthesis;
an elongate gear rod defining a plurality of serrations, each serration being of equal length, the gear rod being attached to the slide member; and
an activation element pivotally connected to the base member, and a driving element extending from the activation element,
wherein, pivotal depression of the activation element causes the driving element to engage one of the serrations on the gear rod and to move the gear rod proximally in relation to the base member, thereby incrementally retracting the sheath from the prosthesis; and further
wherein, the activation element is dimensionally configured in relation to the base member and the gear rod such that proximal movement of the gear rod caused by a single depression of the activation element cannot exceed the length of a single serration.

2. The deployment system of claim 1, further including a locking element, configured to prevent the gear rod from moving distally in relation to the base member.

3. The deployment system of claim 3, wherein the locking element extends substantially perpendicular to the gear rod.

4. The deployment system of claim 3, wherein the locking element defines an opening for permitting passage of the gear rod.

5. The deployment system of claim 3, further including a biasing element configured to urge the locking element in a direction perpendicular to the gear rod.

6. The deployment system of claim 5, wherein the locking element is configured to be manually moved, against the urging of the biasing element, so as to unlock the gear rod and thereby to permit the gear rod to move distally in relation to the base member.

7. The deployment system of claim 1, wherein the slide member is operably attached to the sheath via a retraction member that includes a tubular portion surrounding the core cylinder.

8. The deployment system of claim 1, wherein the base member includes a circular cylinder.

9. The deployment system of claim 8, wherein the slide member includes a piston slideably fitted within the circular cylinder.

10. The deployment system of claim 1, wherein the activation element is biased to an equilibrium position by a leaf spring.

11. The deployment system of claim 10, wherein the leaf spring defines an opening for passage of the gear rod.

12. The deployment system of claim 1, wherein the driving element is flexible along its elongate axis, whereby the driving element can assume a deformed shape to permit the activation element to pivotally rotate while the driving element is engaged with the gear rod.

13. A deployment system for deploying a prosthesis in a duct, comprising:
an elongate catheter for delivering a prosthesis to a predetermined location in the duct;
a prosthesis surrounding the catheter;
a sheath surrounding the prosthesis; and
a handle for deploying the prosthesis, the handle comprising:
a cylinder operably connected to the catheter;
a piston operably connected to the sheath, the piston being moveable in relation to the cylinder, whereby, when the piston is moved proximally in relation to the cylinder, the sheath is withdrawn from the prosthesis;
an elongate gear rod defining a plurality of serrations, each serration being of equal length, the gear rod being attached to the piston; and
a button pivotally connected to the base member;
a leaf spring having first and second ends, the leaf spring being connected at the first end to the cylinder and being in contact with the button at the second end, the leaf spring being configured to urge the button to an equilibrium position;
wherein, pivotal depression of the button inward from the equilibrium position engages the gear rod and moves the gear rod proximally in relation to the cylinder, thereby incrementally retracting the sheath from the prosthesis.

14. The deployment system of claim 13, wherein the button is dimensionally configured in relation to the cylinder and the gear rod such that proximal movement of the gear rod caused by a single depression of the button cannot exceed the length of a single serration.

15. The deployment system of claim 13, wherein the button includes an opening and the second end of the leaf spring is positioned within the opening, the opening being sized to permit the leaf spring to slide within the opening when the button is depressed.

16. The deployment system of claim 13, wherein the button includes a lip configured to engage with the cylinder, whereby the button is held in an equilibrium position under which the leaf spring biases the button radially outwardly.

17. A deployment system for deploying a prosthesis in a duct, comprising:
an elongate catheter for delivering a prosthesis to a predetermined location in the duct;
a prosthesis surrounding the catheter;
a sheath surrounding the prosthesis; and
a handle for deploying the prosthesis, the handle comprising:
a cylinder operably connected to the catheter;
a piston operably connected to the sheath, the piston being moveable in relation to the cylinder, whereby, when the piston is moved proximally in relation to the cylinder, the sheath is withdrawn from the prosthesis;
a button pivotally connected to the base member, the button being in contact with a biasing means for biasing the button to an equilibrium position;
means for controlling distal movement of the piston in relation to the cylinder, whereby said means for controlling permits the piston to move proximally in relation to the cylinder in a series of incremental displacements in which a succeeding incremental displacement is not capable of being unequal to a preceding incremental displacement.

18. The deployment system of claim 17, further including a means for locking the means for controlling against distal movement in relation to the cylinder.

19. A method of retracting a sheath to uncover a prosthesis mounted on a catheter, comprising:
operably attaching the catheter to a base member having a first elongate axis, the base member being positioned proximal to the catheter;
operably attaching the sheath to a slide member having a second elongate axis, the slide member being slideable parallel to the first axis;
repeatedly depressing an activation element that is pivotally attached to the base member;
causing the slide member to move proximally, by no more than the length of a single serration on a gear rod attached to the slide member, with each depression of the activation element; and
withdrawing the activation element to an equilibrium position after each depression;
whereby, the sheath is incrementally moved proximally to uncover the prosthesis.

20. The method of claim 19, further including locking the gear rod against distal movement in relation to the base member.

21. The method of claim 19, wherein withdrawing the activation element after each depression includes withdrawing the activation element under the urging of a leaf spring.

22. The method of claim 19, wherein causing the slide member to move proximally includes engaging the gear rod with a driving element attached to the activation element.

23. A method of retracting a sheath to uncover a prosthesis mounted on a catheter, comprising:
operably attaching the catheter to a cylinder;
operably attaching the sheath to a piston, the piston being slideable within the cylinder;
repeatedly depressing a button that is pivotally attached to the cylinder;
causing the piston to move proximally with each depression of the button in a series of incremental movements, wherein each succeeding incremental movement is equal to each preceding movement;

24. The method of claim 23, further including locking the piston against distal movement after each depression of the button.

25. The method of claim 23, further including withdrawing the activation element to a starting position after each depression.
